(19) United States
(12) Patent Application Publication
(10) Pub. No.: US 2007/0295835 A1
(43) Pub. Date: Dec. 27, 2007

(54) Device and System for Producing Individual Droplets from Liquids Having Different Viscosity in Gaseous and/or Liquid Media

(76) Inventor: Rainer Pommersheim, Mainz (DE)

Correspondence Address: BODNER & O'ROURKE, LLP, 425 BROADHOLLOW ROAD, SUITE 108, MELVILLE, NY, 11747, US

(21) Appl. No.: 10/590,387

(22) PCT Filed: Feb. 23, 2005
(86) PCT No.: PCT/EP05/01892

§ 371 Date: Jun. 22, 2007

(30) Foreign Application Priority Data
Feb. 24, 2004 (DE) 10 2004 009 386.5

(51) Int. Cl. B65D 47/18; A62C 5/00; A61M 11/02; B05B 7/04

(52) U.S. Cl. 239/398; 239/366; 239/311

(57) ABSTRACT

The invention relates to a device by means of which droplets can be produced from liquids having a different viscosity both in gaseous and liquid media. The inventive system is characterized by a nozzle which is devoid of moving parts, which has a modular architecture and which can be used both in gaseous media and in liquids.

The invention relates to a device by means of which droplets from liquids having a different viscosity both in gaseous and liquid media can be produced. The inventive system is characterized by a nozzle which is devoid of moving parts, which has a modular architecture. The liquid is passed in the interior of the nozzle via a capillary. In a gaseous environment the drop separation is accomplished by an air flow passed in the interior of the nozzle concentrically to the central capillary. In gaseous media the droplets are ejected by jets of compressed air which result in an interruption of the liquid flow in the capillary itself.

In technological practice it is frequently required to produce individual droplets from different liquids. The simplest and most widespread method to achieve this resides in spraying by means of suitable nozzles. Such nozzles are offered in commerce in a very large constructive variety. The range reaches from a simple spray head or lawn sprinkler to high-tech developments in the fields of mechanical engineering or paints and varnishes. All of these systems are constructed to produce a spray mist or at least a spray jet formed of innumerable droplets which can, however, neither be influenced individually nor can they be defined more closely.

However, if one wants to produce exactly defined droplets so as to obtain thereby spherical particles by means of chemical or physical hardening, the aforementioned systems are unusable due to their inaccuracy in view of the generated individual droplets. For such purposes systems are applied which are capable of producing precise liquid jets, which are disintegrated subsequently to form individual droplets of defined sizes.

In all of these systems the liquid jets are produced by pressing the liquid starting materials through capillary orifices. Differences merely occur in the methods by means of which these jets are separated into individual droplets.

The methods for this purpose may be classified in two large groups:
1. Methods, in which the liquid jet experiences in addition to its axial movement other movements, such as a rotation or vibration, and
2. Methods, in which the liquid jet experiences no additional movement except for its axial flow movement.

In the first category the jet is disintegrated by centrifugal forces or resonant vibrations, respectively, in the second one by the axial influence of additional media which are, as a rule, gaseous ones. The present invention belongs to the second group.

In technical literature systems serving the production of individual droplets from liquids can be found in numerous places. Only some of them shall be mentioned as examples below.

For example, F. Lim and A. Sun describe in the magazine “Science”, volume 210, pages 908-910, 1980, a method using capillaries at which the drop is separated by an air flow. Thus, one obtains capsule sizes between approximately 200 μm and approximately 2 mm with a very narrow size distribution. However, this publication primarily relates to a method for the encapsulation of cells; a complete laboratory apparatus for the production of droplets is not described therein.

Another method for the production of droplets is the one described in the German patent application 3836894 according to which several capillaries are caused to vibrate, which entails a separation of the liquid jets into individual droplets. In this case, too, the obtained capsules have diameters between approximately 200 μm and approximately 2 mm, while the productivity thereof is clearly higher than that of the above-mentioned nozzles, but with a much wider size distribution. Also, the system needs a readjustment for every new application.

All of these systems always make use of a device for producing droplets which frequently also comprises moving parts. By this, the flexibility is strongly limited, or the expenditure for maintenance and handling increases. Also, they only function in gaseous media, as the droplets always require a distance of fall. Accordingly, they cannot directly be used in liquids.

Another category are the so-called Y-nozzles. They function according to the principle that a liquid column produced in a capillary is interrupted by a pulsed air flow in the interior of the capillary. By this, the droplet is ejected.

Such a system is described, for example, in the application PCT/EP99/01673 (WO 99/47906). The device makes use of a Y-shaped tubular structure. The liquid is pressed through one Y-branch. The liquid column inside the tube is interrupted by an air flow passed via the second Y-branch. According to this invention the structure is designed for an operation in gaseous media.

A similar operating principle is underlying the Japanese application No. 08252913. However, one of the Y-branches is, in this case, sealed by a vibrating plate, while the liquid is supplied by the other one. In this manner the system works without additional air. The drop is ejected by the pressure generated by the vibrating plate. This device, too, is designed for use in gaseous media.

On the basis of this prior art it is the object of the invention to provide a device operating with a nozzle that combines the two aforementioned operating principles, namely, a drop separation achieved both by a concentric air flow in the interior of the nozzle and by an interruption of the liquid flow in the liquid-carrying capillary itself. Moreover, the nozzle is designed for the use both in gaseous and in liquid media.

According to the invention the device or the system, respectively, is subdivided into two portions—the nozzle and the periphery with additional control components serving the media supply and the control of the nozzle.

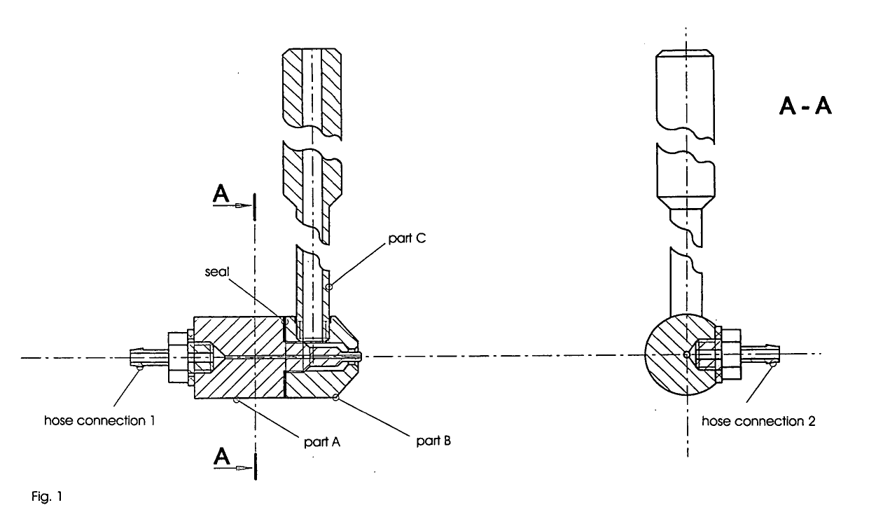

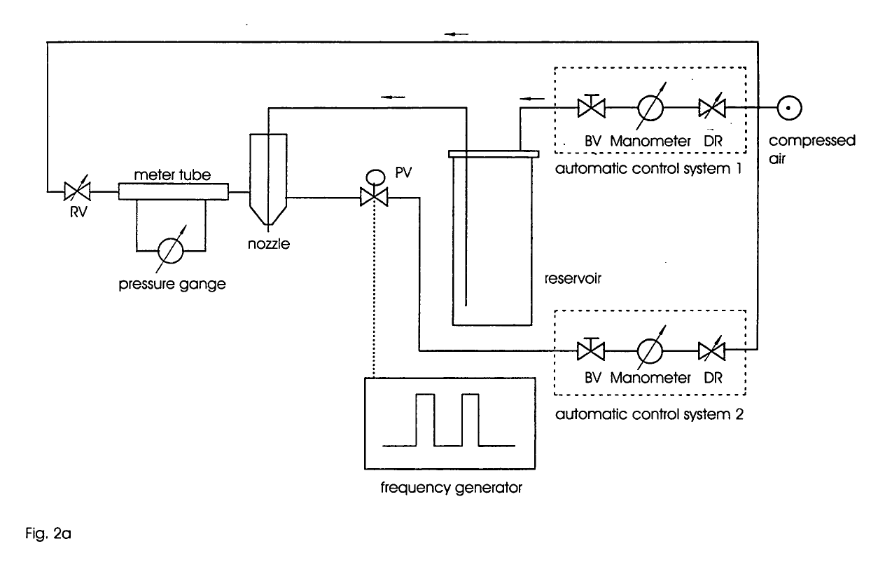

The nozzle is constructed according to FIG. 1. This nozzle may also be applied directly in the liquid, i.e. without any distance of fall for the droplets. This provides for advantages above all with respect to requirements for sterility. Part A is designed such that it forms a capillary by the bore in its interior and the taper in the front part. The liquid is pressed into this capillary via the hose connection 1. A second conduit provided with a hose connection 2 and serving the supply of compressed air is arranged vertically to this bore. If a liquid is now pressed via hose connection 1 through part A interruptions in the liquid flow, which eject the liquid droplets, can be generated by compressed air pulses supplied into the conduit via the hose connection 2. In addition, air can be blown via part C into the interior of part B in a manner concentric to the liquid-carrying capillary (part A). By this, a droplet separation is achieved also without the air pulses supplied via the hose connection 2. The diameter of the droplets obtained has an inverse proportion with respect to the air flow supplied via part C.

Depending on the use of the nozzle, thus, two different operating principles are achieved:
1. In a gaseous medium the nozzle operates such that the droplet separation is achieved by a concentric air flow blown via part C into the interior of part B. Prior to this, hose connection 2 is closed either by a corresponding valve position or mechanically by a sealing element.
2. In a liquid medium part B can be screwed off together with part C, and the outlet opening of part A can be immersed completely in a liquid, e.g. in a precipitating reagent for the produced droplets. As a result of the interruptions in the liquid jet, which are caused by the air pulses supplied via connection 2, the droplets are shot into the precipitating reagent.

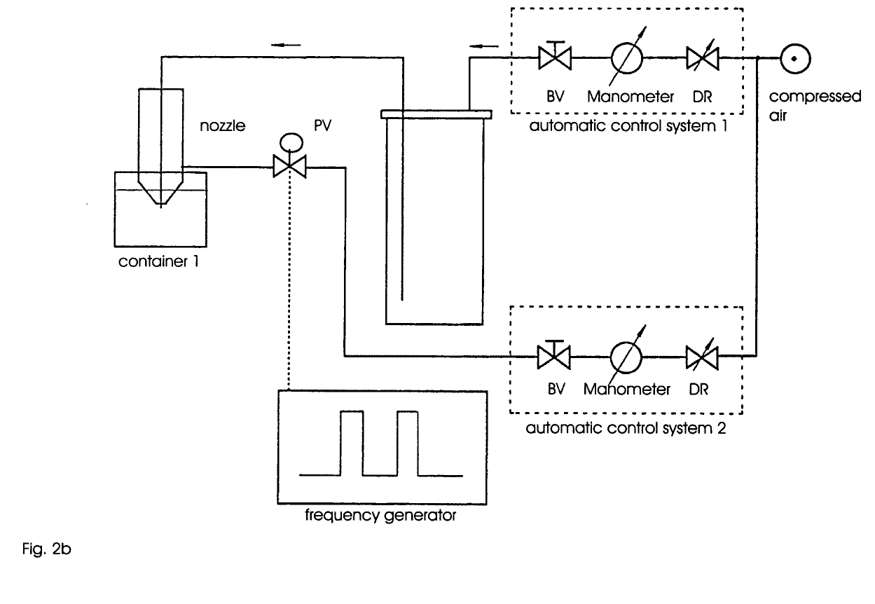

FIG. 2a shows the inventive system comprising the nozzle of FIG. 1 for the use in gaseous media. By actuating the valve PV in a suitable manner connection 2 on the nozzle is closed. Alternatively, hose connection 2 can also be closed with a suitable sealing element.

In this configuration the nozzle operates as follows: A reservoir is supplied with pressure by means of an automatic control system comprising a pressure control valve DR, a manometer and a stop valve BV. This container contains the liquid material from which drops are to be produced. Under the influence of the pressure the liquid is pressed through the capillary of the nozzle. The air flow, which controls the separation of the drops at the capillary, is adjusted by the control valve RV and is measured with a meter tube. A reduced cross-section in the interior of the meter tube generates a pressure difference which depends on the air flow flowing through the tube. This pressure difference is detected by a differential pressure gauge connected to both connection pieces of the meter tube. The higher the air flow, the smaller the obtained droplets. With this configuration also the frequency generator and the valve PV (closure of the second nozzle opening with a sealing element) may be waived.

However, these parts are indispensable when the nozzle is used in liquid media, as is shown in FIG. 2b. In this case, the nozzle outlet opening is completely immersed in the precipitating reagent for the liquid from which droplets are to be produced, which is placed in the container 1. The air controlled by the automatic control system 2 and pulsed by the valve PV and the frequency generator thereby interrupts the liquid flow inside the nozzle and shoots the droplets into the precipitating reagent. The feeding of the liquid from which drops are to be produced through the nozzle is controlled, in correspondence with FIG. 2a, by the automatic control system 1.

1. Device for producing individual droplets from liquids having a different viscosity with a nozzle, characterized in that the material from which droplets are to be produced is pressed by compressed air from at least one container through at least one capillary in the interior of a nozzle and the droplet separation or ejection can be accomplished both by means of an air flow passed concentrically to the capillary in the nozzle and by means of jets of compressed air resulting in an interruption of the liquid flow in the liquid-carrying capillary itself.

2. Device according to claim 1, operating according to a method according to claim 1, characterized in that it comprises one or more of the following main components:
nozzle
reservoir for the material from which droplets are to be produced
automatic control system for supplying the reservoir with compressed air
control elements for controlling the concentric air flow which causes the separation of the droplet in the nozzle
control device and control valve for the jets of compressed air generating the interruptions in the liquid column inside the capillary.

3. Device according to claim 1, characterized in that it operates in accordance with FIG. 2a and/or its components are arranged and/or connected to each other in accordance with FIG. 2a.

4. Device according to claim 1, characterized in that it operates in accordance with FIG. 2b and/or its components are arranged and/or connected to each other in accordance with FIG. 2b.

5. Device according to claim 1, characterized in that liquid droplets can be produced therewith in a gaseous medium by means of a nozzle.

6. Device according to claim 1, characterized in that liquid droplets can be produced therewith in liquid media by means of a nozzle.

7. Device according to claim 1, characterized in that it comprises a nozzle which operates in accordance with FIG. 1 and/or the components of which are set up, arranged and/or connected to each other in accordance with FIG. 1.

8. Device according to claim 1, characterized in that the produced droplets can be precipitated chemically, e.g. by the influence of salts.

9. Device according to claim 1, characterized in that the produced droplets can be precipitated physically, e.g. by a temperature change.

10. Device according to claim 1, characterized in that the precipitated droplets contain the material to be immobilized.
